SEQUENCE LISTING (2 sequences)

SEQ ID NO: 1 - protein, 539 aa, synthetic construct
EYDAVWSKWERDAPAGESPGRAAVVQEMRDCLNNGNPVLNVGASGLTTLPDRLPPHITTLVIPDNNLTSLPELPEGLRELEVSGNLQLTSLPSLPQGLQKLWAYNNWLASLPTLPPGLGDLAVSNNQLTSLPEMPPALRELRVSGNNLTSLPALPSGLQKLWAYNNRLTSLPEMSPGLQELDVSHNQLTRLPQSLTGLSSAARVYLDGNPLSVRTLQALRDIIGHSGIRIHFDMAGPSVPREARALHLAVADWLTSAREGEAAQADRWQAFGLEDNAAAFSLVLDRLRETENFKKDAGFKAQISSWLTQLAEDAALRAKTFAMATEATSTCEDRVTHALHQMNNVQLVHNAEKGEYDNNLQGLVSTGREMFRLATLEQIAREKAGTLALVDDVEVYLAFQNKLKESLELTSVTSEMRFFDVSGVTVSDLQAAELQVKTAENSGFSKWILQWGPLHSVLERKVPERFNALREKQISDYEDTYRKLYDEVLKSSGLVDDTDAERTIGVSAMDSAKKEFLDGLRALVDEVLGSYLTARWRLN

SEQ ID NO: 2 - protein, 78 aa, synthetic construct
ATNLSRVAGLEKQLAIELKVKQGAENMIQTYSNGSTKDRKLLLTAQQMLQDSKTKIDIIRMQLRRALQAGQLENQAAP

(19) United States
(12) Patent Application Publication
(10) Pub. No.: US 2025/0019674 A1
(43) Pub. Date: Jan. 16, 2025

(54) PROTEIN DEGRADATION SYSTEM, COMPONENTS, AND METHODS

(71) Applicant: North Carolina State University, Raleigh, NC (US)

(72) Inventors: Marcela Pierce, Raleigh, NC (US); Linzhou Huang, Raleigh, NC (US)

(21) Appl. No.: 18/771,308

(22) Filed: Jul. 12, 2024

Related U.S. Application Data
(60) Provisional application No. 63/513,490, filed on Jul. 13, 2023.

(51) Int. Cl. C12N 9/12; C12N 9/10

(52) U.S. Cl. CPC C12N 9/12; C12N 9/104; C07K 2319/30; C07K 2319/70; C12N 2840/007; C12Y 203/02; C12Y 207/11013

(57) ABSTRACT

The present disclosure provides components and systems for targeted protein degradation in cells (e.g., in plant cells). In particular, the present disclosure provides a protein containing the Leucine-Rich Repeat (LRR) and novel E3 ligase (NEL) domains of SspH1 for use with Homology Region 1b (HR1b) domain of human PKN1 for degrading target proteins.

### CROSS-REFERENCE TO RELATED APPLICATIONS

This application claims the benefit of U.S. Provisional Application No. 63/513,490, filed Jul. 13, 2023, the content of which is herein incorporated by reference in its entirety.

### GOVERNMENT SUPPORT

This invention was made with government support under grant number MCB1909923 awarded by the National Science Foundation. The government has certain rights in the invention.

### FIELD

The present disclosure provides components and systems for targeted protein degradation in cells (e.g., in plant cells). In particular, the present disclosure provides a protein containing the Leucine-Rich Repeat (LRR) and novel E3 ligase (NEL) domains of SspH1 for use with Homology Region 1b (HR1b) domain of human PKN1 for degrading target proteins.

### SEQUENCE LISTING STATEMENT

The content of the electronic sequence listing titled NCSU-42109-202.xml (Size: 3,364 bytes; and Date of Creation: Jul. 12, 2024) is herein incorporated by reference in its entirety.

### BACKGROUND

Inducible protein knockdowns are excellent tools to test the function of essential proteins in short time scales and to capture the role of proteins in dynamic events. Targeting proteins for degradation by hijacking endogenous ubiquitin-proteasome systems has been exploited by cell biologists. Unfortunately, there are very few examples of systems suitable for use in plants. Most systems utilize a ligand and ligand-binding domains to recruit the target protein to an E3 ubiquitin ligase (E3 ligase), while others take advantage of instability domains, or degrons, to trigger protein degradation. Several common ligand-based systems approaches destroy or sequester proteins by exploiting plant biological mechanisms such as the activity of photoreceptors for optogenetics or auxin-mediated ubiquitination in auxin degrons. As light and auxins are very strong signals for plant cells, these approaches are not applicable for plants.

Many cellular processes in plant cells are extremely dynamic. Capturing the dynamics of these processes by functional studies of target proteins requires perturbation systems with high-temporal resolution. In addition, disruption of essential proteins in plants is hampered by the lethality of loss-of-function mutants, a phenomenon very common in pathways that involve the endomembrane system. Reliable systems for inducible protein depletion to test the function of essential proteins in short time scales and to capture dynamic events, however, are not available in plants. Thus, there is a need for protein degradation systems suitable for protein depletion, especially inducible protein depletion, in plants.

### SUMMARY

Embodiments of the present disclosure include systems for targeted protein degradation in a cell. In some embodiments, the systems comprise a first polypeptide comprising Leucine-Rich Repeat (LRR) domain and novel E3 ligase (NEL) catalytic domain of SspH1, or a nucleic acid encoding thereof and a second polypeptide comprising Homology Region 1b (HR1b) domain of human PKN1, or a nucleic acid encoding thereof.

In some embodiments, the first polypeptide or the second polypeptide further comprise a targeting moiety configured to specifically bind a protein of interest. In some embodiments, the targeting moiety directly binds the protein of interest. In some embodiments, the targeting moiety is an antibody or fragment thereof to the protein of interest. In some embodiments, the targeting moiety indirectly binds the protein of interest. In some embodiments, the targeting moiety is a first part of a specific binding pair and the protein of interest comprises a second part of the specific binding pair.

In some embodiments, the first polypeptide or the second polypeptide is linked to a third polypeptide comprising the protein of interest. In some embodiments, the third polypeptide is linked to the first polypeptide or the second polypeptide by an amino acid linker.

In some embodiments, the nucleic acid encoding the first polypeptide and/or the nucleic acid encoding the second polypeptide are operably linked to a tissue or cell specific promoter. In some embodiments, the nucleic acid encoding the first polypeptide and/or the nucleic acid encoding the second polypeptide are operably linked to a regulatable promoter.

In some embodiments, the nucleic acid encoding the first polypeptide and/or the nucleic acid encoding the second polypeptide are provided on a vector or plasmid.

In some embodiments, the first polypeptide or second polypeptide are heterologous to the cell.

In some embodiments, the cell is a non-mammalian cell. In some embodiments, the cell is a plant cell.

In some embodiments, the protein of interest is a membrane protein, a receptor, a hormone, a transport protein, a transcription factor, a cytoskeletal protein, an extracellular matrix protein, a signal-transduction protein, or an enzyme.

Embodiments of the present disclosure further include cells comprising the disclosed system. In some embodiments, the cell is a non-mammalian cell. In some embodiments, the cell is a plant cell.

Embodiments of the present disclosure also include plants comprising the disclosed system.

Embodiments of the present disclosure additionally include fusion proteins comprising: a protein of interest; and Homology Region 1b (HR1b) domain of human PKN1 or a polypeptide comprising Leucine-Rich Repeat (LRR) domain and novel E3 ligase (NEL) catalytic domain of SspH1. In some embodiments, the protein of interest is linked to the Homology Region 1b (HR1b) domain of human PKN1 or a polypeptide comprising Leucine-Rich Repeat (LRR) domain and novel E3 ligase (NEL) catalytic domain of SspH1 by an amino acid linker.

Also provided are methods for degrading a protein of interest in a cell. In some embodiments, the methods comprise contacting the cell with a system disclosed herein, wherein the system induces degradation of the protein of interest. In some embodiments, the cell is a non-mammalian cell. In some embodiments, the cell is a plant cell. In some embodiments, the cell is in vitro or ex vivo. In some embodiments, the cell is in vivo.

In some embodiments, method comprises administering the system to a host organism. In some embodiments, the host organism is a plant.

Other aspects and embodiments of the disclosure will be apparent in light of the following detailed description and accompanying figures.

### BRIEF DESCRIPTION OF THE DRAWINGS

FIGS. 1A-1C: Design of the plant protein degron system. (FIG. 1A) The degron system is based on the interaction between the Salmonella secreted protein H1 (SspH1) and the human protein kinase N1 (PKN1). The interaction between the HR1b domain of PKN1 and the Leucine-Rich repeat (LRR) domain of SspH1 results in protein binding and ubiquitination by the NEL domain. The HR1b domain is fused to a target protein to recruit the truncated LRR-NEL fragment and induce ubiquitination and target protein degradation. (FIG. 1B) Protein chimeras used in this study. The LRR-NEL fragment was fused to mCherry and the HR1b domain was fused to GFP. A C492A substitution in the NEL domain corresponds to a catalytically inactive control and the R181A and R185A mutations in HR1b correspond to non-interacting controls. HA, Hemagglutinin tag; LRR, Leucin-rich repeat; GFP, green fluorescent protein. (FIG. 1C) Transient expression of degron components in Nicotiana benthamiana. Infiltrated leaves were imaged by confocal microscopy to detect mCherry or GFP signal. The merged image with brightfield acquisition is included. Scale: 40 μm.

FIGS. 2A-2F: HA-LRR-NEL-mCherry targets HR1b-GFP for efficient degradation in plants. (FIG. 2A) Co-immunoprecipitation (Co-IP) assay using crude extracts from agroinfiltrated Nicotiana leaves confirmed the interaction between HR1b-GFP and both HA-LRR-NEL-mCherry and HA-LRR-NEL^{C492A}-mCherry. Protein extracts from independent transformations were mixed as indicated and binding proteins were immunoprecipitated with anti-GFP and detected by immunoblot. (FIG. 2B) Confocal microcopy of leaves transiently co-transformed with HR1b-GFP or HR1b^{R181/185A}-GFP and HA-LRR-NEL-mCherry or HA-LRR-NEL^{C492A}-mCherry. Scale bars: 100 μm. All images were taken with the same microscope settings. (FIG. 2C) The NEL E3 ligase chimera targets HR1b-GFP for degradation in plants. Equal amounts of Agrobacterium strains carrying each indicated construct were co-infiltrated in Nicotiana leaves and recombinant proteins were detected by immunoblot. (FIG. 2D) HR1b stability is dependent on the NEL E3 ligase chimera. Equal amounts of Agrobacterium strains carrying the HR1b-GFP construct were co-infiltrated with increasing amounts Agrobacteria carrying the HA-LRR-NEL-mCherry construct. Expressed proteins were detected by immunoblot. Co-transformation with Flag-RFP-Myc was used as a control in (FIGS. 2C and 2D). Total RNA was extracted from the agroinfiltrated Nicotiana leaves and RT-PCR was performed to detect HR1b-GFP and HR1b^{R181/185A}-GFP transcripts. (FIG. 2E) The NEL E3 ligase chimera targets HR1b-GFP for degradation in Arabidopsis. Arabidopsis plants were sequentially transformed with the indicated constructs and roots were imaged by confocal microscopy. All transgenes were driven by the UBIQUITIN10 promoter and all images were captured with the same microscope settings. Scale bar: 100 μm. (FIG. 2F) HR 1b-GFP is unstable in plants expressing the NEL E3 ligase chimera. Proteins were extracted from transgenic seedlings (FIG. 2E) and detected by immunoblot. Actin was used as endogenous control. HA-LRR-NEL-mCherry and HA-LRRC^{492A}-NEL-mCherry proteins were detected with anti-HA monoclonal antibody; HR1b-GFP, HR1b^{R181/185A}-GFP and GFP were detected with anti-GFP monoclonal antibody; Flag-GFP-Myc was detected with anti-Myc monoclonal antibody and Arabidopsis actin was detected with anti-Actin polyclonal antibody.

FIGS. 3A-3F: Robust degradation of HR1b-tagged plant proteins by HA-LRR-NEL-mCherry. Degradation of a cytoplasmic VPS34-GFP-HR1b (FIGS. 3A, 3B) or nuclear HR1b-GFP-RHD6 (FIGS. 3C-3F) fusions by the NEL E3 ligase chimera (HA-LRR-NEL-mCherry, FIGS. 3A-3D) or a nuclear localized ligase (3xNLS-HA-LRR-NEL-mCherry, FIGS. 3E-3F). Nicotiana leaves were infiltrated with Agrobacterium carrying each pair of expression cassettes in the same plasmid. The catalytically inactive ligase (HA-LRR-NEL^{c492A}-mCherry) and mutant HR1b peptide (HR1b^{R181/185A}) were used as controls and all plasmids were infiltrated using equal amounts of Agrobacteria. GFP and mCherry signals were detected by confocal microscopy and the merged image including brightfield acquisition is shown. Scale: 100 μm (FIGS. 3A, 3C, 3E). Protein abundance from similar transformations was detected by immunoblot and co-transformation with Flag-RFP-Myc was used as a transformation control (FIGS. 3B, 3D, 3F). RT-PCR was used to confirm expression of the HR1b and HR1b^{R181/185A} fusions. Proteins fused to HA and GFP tags were detected by anti-HA and anti-GFP monoclonal antibodies, respectively. The Flag-RFP-Myc was detected by anti-Myc monoclonal antibody.

FIGS. 4A-4B: Establishment of a Dexamethasone-inducible degron system for plants. (FIG. 4A) A Dexamethasone (DEX)-inducible system was developed by controlling the expression of the NEL E3 ligase chimera with the GVG transactivation system. The GAL4-VP16-GR (GVG) transcription factor is retained in the cytosol in the absence of DEX due to a large inhibitory complex containing HSP70, HSP90 and other proteins. DEX treatment results in its binding to the ligand binding domain of the glucocorticoid receptor (GR), dissociation of GVG from the HSP complex and movement into the nucleus. The GVG transcription factor then binds to the GAL4 upstream activating sequence (6XUAS) and triggers the transcription of the NEL E3 ligase coding sequence. Once the ligase is translated, it can bind and ubiquitinate HR1b-bearing targets. (FIG. 4B) DEX-induced degradation of HR1b-GFP fusions by the NEL E3 ligase chimera. Nicotiana leaves were infiltrated with Agrobacterium carrying DEX-inducible ligases (HA-LRR-NEL-mCherry or HA-LRR-NEL^{c492A}-mCherry) together with the HR1b-GFP or HR1b^{R181/185A}-GFP in the same plasmid. 100 μM DEX was applied 48 h after transformation by immersion of leaf petioles, and proteins were extracted up to 6 h later. All plasmids were infiltrated using equal amounts of Agrobacteria. Myc-GVG-HA was used as a control. HR1b-GFP and HR1b^{R181/185A}-GFP transcripts were detected by RT-PCR.

FIGS. 5A-5B: Degradation of endogenous proteins with the DEX-induced degron system. DEX-induced degradation of HR1b fusions to VPS34 (A) and RHD6 (B) by the NEL E3 ligase chimera. (FIG. 5A) Nicotiana leaves were infiltrated with Agrobacterium carrying DEX-inducible ligases (HA-LRR-NEL-mCherry or HA-LRR-NEL^{c492A}-mCherry) together with the target fusions (VPS34-GFP-HR 1b or VPS34-GFP-HR1b^{R181/185A}-GFP) in the same plasmid. (FIG. 5B) Leaves were infiltrated with Agrobacterium carrying DEX-inducible ligases together with the HR1b-GFP-RHD6 or HR1b^{R181/185A}-GFP-RHD6 in the same plasmid. 100 μM DEX was applied 48 h after transformation by immersion of leaf petioles, and proteins were extracted up to 6 h later. Detection of Myc-GVG-HA was used as a control. HR1b- and HR1b^{R181/185A}-containing transcripts were detected by RT-PCR.

FIG. 6: The GRBD domain is not sufficient to inhibit the LRR-NEL fragment in the absence of Dexamethasone. Fluorescence microscope analysis of the expression of GRBD-LRR-NEL-mCherry or GRBD-LRR-NEL-GRDB-mCherry chimeras combined with HR1b-GFP or HR1b^{R181/185A}-GFP in Nicotiana benthamiana. Both the E3 ligase and the target protein expression cassettes were cloned in the same plasmid. GFP and mCherry signals were captured in an epifluorescence microscope. All the images were taken with the same settings. Scale: 256 μm.

FIG. 7: The hERBD is insufficient to inhibit the E3 ligase activity of the LRR-NEL fragment in the absence of β-estradiol. Fluorescence microscopy of the expression of hERBD-LRR-NEL-mCherry and HR1b-GFP (top panel) and HR1b^{R181/185A}-GFP (bottom panel) in Nicotiana benthamiana. Both the E3 ligase and the target protein expression cassettes were cloned in the same plasmid. All the images were taken with the same settings. Scale: 256 μm.

FIGS. 8A-8B: Activation of nb-E3-DART with a HR1b peptide. (FIG. 8A) A nanobody(nb)-E3-DART fusion is autoinhibited in the absence of HR1b peptide. Addition of free HR1b peptide to nb-E3-DART results in ligase activation and ubiquitination of free GFP. (FIG. 8B) GFP degradation was observed when nb-E3-DART and GFP fusions are co-expressed with free HR1b peptide but not without. Nicotiana leaves were transiently transformed with the indicated constructs with Agrobacterium infiltration. GFP and mCherry signal was detected by epifluorescence microscopy.

### DETAILED DESCRIPTION

Embodiments of the present disclosure provide systems for targeted protein degradation in cells (e.g., in plant cells), components of the system, and methods of using the system. The present systems harness the E3 ubiquitin ligase catalytic activity of SspH1 and the SspH1-binding activity of the Homology Region 1b (HR1b) domain from PKN1.

As disclosed herein, a chimeric protein containing the Leucine-Rich Repeat (LRR) and novel E3 ligase (NEL) domains of SspH1 efficiently, specifically, and dependently degraded protein fusions of varying sizes containing HR1b, due to the HR1b-LRR interaction. The overall system utilizing this chimeric protein provided a robust mode of protein stability control in plant cells. The HR1b domain was functional at either N or C termini of target proteins thus providing flexibility for synthetic fusion constructs of target proteins. Two amino acid residues, R181 and R185 within the HR1b domain in SspH1, shown to be required for HR1b-LRR interactions, can offer precise negative controls in cell biology experiments to demonstrate specificity of observed phenotypes. HR1b activates LRR-NEL activity (e.g., as an ON/OFF switch) for target protein degradation even when not directly fused to the target protein. There was no evidence of morphological or developmental defects in Arabidopsis lines expressing the chimeric NEL E3 ligase, suggesting minimal to no off-target effects on endogenous proteins. Given its robust catalytic activity, it was likely that the chimeric NEL E3 ligase folded correctly at the temperatures in which plants are grown (22° C.), more than 15 degrees lower than its normal temperature. Furthermore, SspH1 could recruit plant E2 ubiquitin-conjugating enzymes (E2s) efficiently, as demonstrated by the strong degradative activity of HR1b targets in Nicotiana and Arabidopsis.

Target protein degradation was induced by transcriptional control of the chimeric E3 ligase using a glucocorticoid transactivation system and target protein depletion was detected as early as 3 hours after induction in Nicotiana. There is only one other example of inducible protein degradation in plants which used the deGradFP in Arabidopsis but protein degradation was only observed 24 hours after induction. The present system could be used to study the loss of any plant protein with high temporal resolution and may become an important tool in plant cell biology.

In the present systems, compared to other degron designs, a single protein (LRR-NEL) provides target recruitment and E3 ligase functions. The E3 ligase does not compete with native proteins for establishment of the E3 ligase complex.

Overall, the disclosed systems including the NEL-LRR fragment and the HR1b degron represent a functional and reliable protein degron system, particularly suitable for protein depletion in plants.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. As used herein, comprising a certain sequence or a certain SEQ ID NO usually implies that at least one copy of said sequence is present in recited peptide or polynucleotide. However, two or more copies are also contemplated. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “antibody” or “immunoglobulin,” as used herein, refers to a protein that is found in blood or other bodily fluids of vertebrates, which is used by the immune system to identify and neutralize foreign objects, such as bacteria and viruses. Typically, an antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

As used herein, when an antibody or other entity (e.g., antigen binding domain or targeting moiety) “specifically recognizes” or “specifically binds” an antigen, epitope, or protein, it preferentially recognizes the antigen, epitope, or protein in a complex mixture of proteins and/or macromolecules, and binds the antigen, epitope, or protein with affinity which is substantially higher than to other entities or proteins not displaying the antigen or epitope. In this regard, “affinity which is substantially higher” means affinity that is high enough to enable detection of an antigen, epitope, or protein which is distinguished from entities using a desired assay or measurement apparatus. Typically, it means binding affinity having a binding constant (Kₐ) of at least 10⁻⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the present disclosure. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)2 fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. Peptides and polypeptides include proteins such as binding proteins, receptors, and antibodies. The terms “polypeptide” and “protein” are used interchangeably herein.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The term “contacting” as used herein refers to bring or put in contact, to be in or come into contact. The term “contact” as used herein refers to a state or condition of touching or of immediate or local proximity. Contacting to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the proteins or systems of the disclosure into a subject by a method or route which results in at least partial localization to a desired site. Administration can use any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. SYSTEM FOR TARGETED PROTEIN DEGRADATION

Disclosed herein are systems for targeted protein degradation in a cell. The systems include a first polypeptide comprising Leucine-Rich Repeat (LRR) domain and novel E3 ligase (NEL) catalytic domain of SspH1, or a nucleic acid encoding thereof and a second polypeptide comprising Homology Region 1b (HR1b) domain of human PKN1, or a nucleic acid encoding thereof. The HR1b domain of the second polypeptide, when bound to the LRR portion of the first polypeptide, facilitates activation of the NEL catalytic domain, e.g., to direct degradation and transfer ubiquitin protein to the protein of interest.

In some embodiments, the first polypeptide comprises a Leucine-Rich Repeat (LRR) domain and novel E3 ligase (NEL) catalytic domain of SspH1 having SEQ ID NO: 1. In some embodiments, the first polypeptide comprises an amino acid sequence having one or more additions, substitutions or deletions as compared to SEQ ID NO:1. For example, the first polypeptide may comprise an amino acid sequence having at least 70% identity (e.g., at least 75%, a least 80%, at least 85%, at least 90%, at least 95%, at least 97%, at least 98%, at least 95%) identity to SEQ ID NO: 1.

In some embodiments, the second polypeptide comprises a Homology Region 1b (HR1b) domain of human PKN1 having SEQ ID NO: 2. In some embodiments, the second polypeptide comprises an amino acid sequence having one or more additions, substitutions or deletions as compared to SEQ ID NO:2. For example, the second polypeptide may comprise an amino acid sequence having at least 70% identity (e.g., at least 75%, a least 80%, at least 85%, at least 90%, at least 95%, at least 97%, at least 98%, at least 95%) identity to SEQ ID NO: 2.

Either the first polypeptide or the second polypeptide may comprise a targeting moiety configured to specifically bind a protein of interest. In some embodiments, the first polypeptide comprises a targeting moiety to bind the protein of interest and the second polypeptide, upon association with the first polypeptide, facilitates activation of the NEL to the protein of interest. In some embodiments, the second polypeptide comprises a targeting moiety to bind the protein of interest and upon association of the second polypeptide with the first polypeptide the NEL is activated to the protein of interest.

The targeting moiety facilitates engagement between the protein of interest and the disclosed protein degradation system. A targeting moiety can be any agent capable of specifically binding or interacting with the desired protein of interest. The targeting moiety may be a polypeptide, glycoprotein, nucleic acid, small molecule, carbohydrate, lipid, etc., for example ligands, such as proteins, antibodies, antibody fragments, and the like.

The targeting moiety can be a nucleic acid (e.g., an aptamer, Spiegelmer®, etc.) that binds to a cell type specific marker. In general, an aptamer is an oligonucleotide (e.g., DNA, RNA, or an analog, derivative, or combination thereof) that binds to a particular target, such as a protein of interest. The targeting moiety may be a naturally occurring or synthetic ligand for a target protein. The targeting moiety may be an antibody or any characteristic fragment thereof. Synthetic binding proteins such as Affibodies®, Nanobodies™, AdNectins™, Avimers™, etc., may be used. Peptide and non-antibody targeting moieties be identified, e.g., using procedures such as phage display (e.g., RGD peptides, NGR peptide, and transferrin LHRH). This widely used technique has been used to identify cell specific ligands for a variety of different targets. The small molecules may include synthetic or natural molecules which target specific binding partners (e.g., folate, galactose).

In some embodiments, the targeting moiety directly binds the protein of interest, such as for example, an antibody, antibody fragment or a natural ligand or binding partner of the protein of interest, as described above.

In some embodiments, the targeting moiety indirectly binds the protein of interest. For example, the targeting moiety is a first part of a specific binding pair and the protein of interest comprises a second part of the specific binding pair. A binding pair refers to a pair of molecules comprising a binding member and a binding partner which have particular specificity for each other and under normal conditions bind to each other in preference to binding to other molecules. The interaction of the binding pair is typically non-covalent, but may also result in formation of a covalent bond. The binding member and binding partner may comprise a part of a larger molecule.

The binding pair may include protein:protein binding pairs (e.g., protein:antibody, biotin:avidin), protein:polynucleotide binding pairs, protein:carbohydrate binding pairs, protein:small molecule binding pairs, polynucleotide:polynucleotide binding pairs, and the like. Examples of a specific binding pair include an antibody and an antigen, biotin and avidin or streptavidin, a ligand and a receptor, a lectin and a carbohydrate, an enzyme and a cofactor or substrate, oppositely charged ionic groups, redox/electrochemical groups, a chelating group and its binding partner, or a nucleic acid molecule capable of hybridizing to a complementary nucleic acid sequence.

In some embodiments, the binding pair comprises functional groups that react to form a covalent bond. For example, functional groups that facilitate bioconjugation reactions (e.g., thiol conjugation reactions, amine-modified DNAs with carbonyl functional groups, and the like). For exemplary pairs see Kalia J, Raines RT. Curr Org Chem. 2010;14(2):138-147, Mukesh Digambar Sonawane, Satish Balasaheb Nimse, Journal of Chemistry, vol. 2016, Article ID 9241378, 19 pages, 2016, and Bioconjugate Techniques, Ed. Hermanson, GT, Academic Press, 1996, Pages 727-728, ISBN 9780123423351, each incorporated herein by reference in its entirety.

The binding pair may be a so-called split system. Split systems include, but are not limited to, intein, MS2, or SunTag based systems.

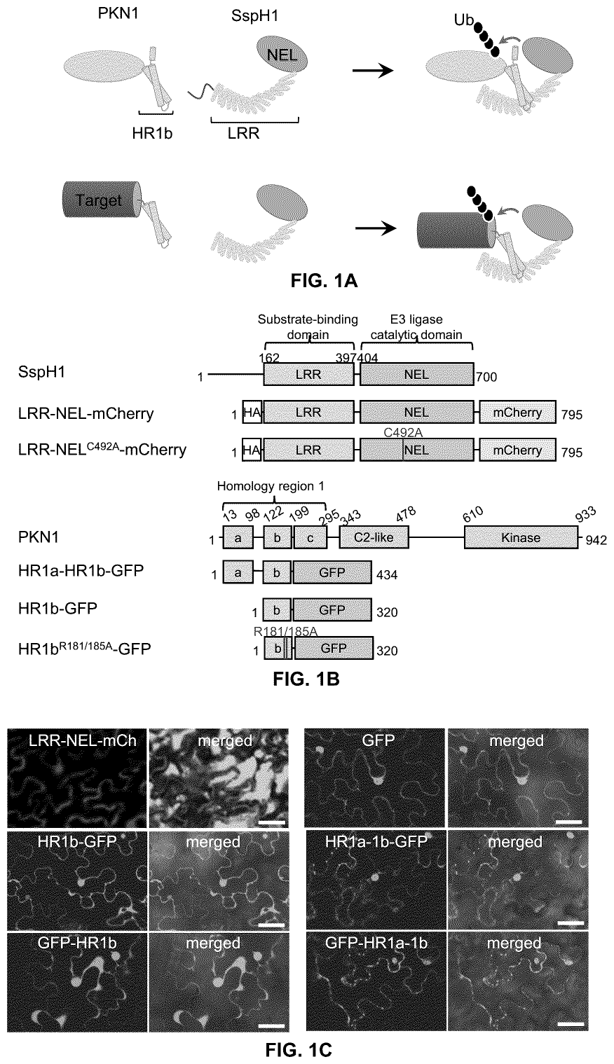

In some embodiments, the first polypeptide or the second polypeptide is linked to a third polypeptide comprising the protein of interest. Thus, upon interaction of the first polypeptide with the second polypeptide the system is activated to degrade the third polypeptide and the protein to which it is linked. For example, as shown in FIG. 1A, when the protein of interest (“Target”) is linked to the second polypeptide, upon interaction with the first polypeptide, the protein of interest is tagged for degradation. The first polypeptide or the second polypeptide may be linked to the protein of interest in any orientation, e.g., N-terminus to N-terminus, C-terminus to C-terminus, N-terminus to C-terminus, or C-terminus to N-terminus.

The invention is not limited by the protein of interest. The protein of interest may comprise a membrane protein, a receptor, a hormone, a transport protein, a transcription factor, a cytoskeletal protein, an extracellular matrix protein, a signal-transduction protein, or an enzyme.

In some embodiments, the protein of interest is an essential protein, such that a targeted degradation strategy in certain cell or cell types, by mechanisms described elsewhere herein, allows study in specific locations without risking the overall health of the organism.

In some embodiments, the proteins of interest may be members of developmental, reproductive, or response pathways. For example, the system may be used to degrade a “kill switch” protein, which when degraded, induces the death in the organism, cell, or organ. In plants, the protein of interest may be a pollen essential protein and confer cytoplasmic male sterility. The protein of interest can be a protein, e.g., CENH3, involved in centromere function. The protein of interest may be a stress response pathway protein. Stress responses are often controlled by protein degradation and the proposed system would facilitate manipulation of stress responses by certain cells, organs, or overall organisms.

In some embodiments, the protein of interest may be a disease-associated protein. A disease-associated protein includes proteins expressed at an abnormal level or in an abnormal form in cells obtained from a disease-affected organism as compared with tissues or cells obtained from an organism not affected by the disease. A disease-associated protein also encompasses pathogenicity-related effector proteins. Pathogenicity-related effector proteins are proteins which allow successful colonization of a pathogen or pest (e.g., a bacterium, virus, fungus, protozoa, nematodes or any other non-bacterial or fungal pest) in the host organism. Effector proteins can be or serve as, for example, virulence factors, pathogen-associated molecular patterns (PAMPs), toxin proteins, elicitors, or degrading enzymes.

The third polypeptide comprising the protein of interest may be appended to the first polypeptide or the second polypeptide by an amino acid linker. The linker may have any of a variety of amino acid sequences. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. Small amino acids, such as glycine and alanine, are generally used in creating a flexible peptide. A variety of different linkers are commercially available and are considered suitable for use, including but not limited to, glycine-serine polymers, glycine-alanine polymers, and alanine-serine polymers.

As such the disclosure also provides a fusion protein comprising a protein or polypeptide of interest and Homology Region 1b (HR1b) domain of human PKN1 or a polypeptide comprising Leucine-Rich Repeat (LRR) domain and novel E3 ligase (NEL) catalytic domain of SspH1. In some embodiments, the polypeptide of interest is linked to the Homology Region 1b (HR1b) domain of human PKN1. In some embodiments, the polypeptide of interest is linked to the polypeptide comprising Leucine-Rich Repeat (LRR) domain and novel E3 ligase (NEL) catalytic domain of SspH1. In some embodiments, the polypeptide of interest is linked to the Homology Region 1b (HR1b) domain of human PKN1 or a polypeptide comprising Leucine-Rich Repeat (LRR) domain and novel E3 ligase (NEL) catalytic domain of SspH1 by a linker, as described above. In some embodiments, the polypeptide of interest is linked to the Homology Region 1b (HR1b) domain of human PKN1 or a polypeptide comprising Leucine-Rich Repeat (LRR) domain and novel E3 ligase (NEL) catalytic domain of SspH1 by an amino acid linker.

In some embodiments, the first polypeptide comprising Leucine-Rich Repeat (LRR) domain and novel E3 ligase (NEL) catalytic domain of SspH1 and/or the second polypeptide comprising Homology Region 1b (HR1b) domain of human PKN1, or a nucleic acid encoding thereof are heterologous to the cell. Thus, the system facilitates target degradation in cells without interfering with the endogenous protein degradation systems of the cell of interest. The system also facilitates target degradation by utilizing other components of the endogenous protein degradation system of the cell, e.g., the E2 ubiquitin conjugation enzymes.

Suitable cells include, but are not limited to: bacterial cell; an archaeal cell; a eukaryotic cell; a cell of a single-cell eukaryotic organism; a plant cell; a protozoa cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell of a vertebrate animal (e.g., a fish, an amphibian, a reptile, a bird); and the like. Any type of cell may be of interest. In some cases, the cell is a cell that does not originate from a natural organism (e.g., the cell can be a synthetically made cell; also referred to as an artificial cell). In some embodiments, the cell is a non-mammalian cell (e.g., a prokaryotic cell, a plant cell, an insect cell, a fungal cell). As such, the disclosure further provides cells comprising the disclosed systems.

Most engineered degradation systems use plant proteins, domains, and/or pathways and are not suitable for use in plants due to interference with the endogenous protein degradation systems. However as disclosed herein, the systems provide utility for targeted protein degradation in a plant or plant cell. In select embodiments, the cell is a plant cell. Non-limiting examples of plant cells include cells from: plant crops, fruits, vegetables, grains, soybean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, seaweeds (e.g., kelp), and the like. The cells can be root cells, leaf cells, cells of the xylem, cells of the phloem, cells of the cambium, apical meristem cells, parenchyma cells, collenchyma cells, sclerenchyma cells, and the like.

The plant cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

As such, the disclosure also provides plant cells and plants engineered to comprise the disclosed systems.

Cells may be from established cell lines or they may be primary cells, where “primary cells,” “primary cell lines,” and “primary cultures” are used interchangeably herein to refer to cells and cells cultures that have been derived from an organism and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

The nucleic acids encoding the components of the disclosed protein degradation system may be any nucleic acid including DNA, RNA, or combinations thereof. In some embodiments, the nucleic acids comprise messenger RNAs, vectors, or any combination thereof.

In some embodiments, the first polypeptide comprising Leucine-Rich Repeat (LRR) domain and novel E3 ligase (NEL) catalytic domain of SspH1 and the second polypeptide comprising Homology Region 1b (HR1b) domain of human PKN1 are encoded by a single nucleic acid (e.g., a single vector). In some embodiments, the first polypeptide and the second polypeptide are encoded by different nucleic acids (e.g., multiple mRNAs or two or more vectors).

In certain embodiments, engineering the system for use in various cells may involve codon-optimization or other modification (e.g., to include an appropriate nuclear localization signal (NLS) or purification tag). It will be appreciated that changing native codons to those most frequently used in the target cells allows for maximum expression of the system proteins. Such modified nucleic acid sequences are commonly described in the art as “codon-optimized.” In some embodiments, the nucleic acid sequence is considered codon-optimized if at least about 60% (e.g., 65%, 70%, 75%, 80%, 85%, 90%, 95%, or 98%) of the codons encoded therein are engineered to preferred codons.

The present disclosure also provides for DNA segments encoding the polypeptides and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

To construct cells that express the present system, expression vectors for stable or transient expression of the present system may be constructed via conventional methods and introduced into cells. For example, nucleic acids encoding the components of the present system may be cloned into a suitable expression vector, such as a plasmid or a viral vector in operable linkage to a suitable promoter.

Nucleic acids of the present disclosure can comprise any of a number of promoters known to the art, wherein the promoter is constitutive, regulatable or inducible, cell type specific, tissue/organ-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), 35S promoter of the Cauliflower Mosaic Virus (CaMV 35S), Ubiquitin (Ubi-1) of maize, promoters derived from the Mirabilis Mosaic Virus (MMV) and Strawberry Vein Banding Virus, actin gene from rice (Act1), and the like.

Alternatively, any regulatable promoter may be used, such that its expression can be modulated within a cell. Moreover, inducible expression can be accomplished by placing the nucleic acid encoding such a molecule under the control of an inducible promoter/regulatory sequence. Promoters well known in the art can be induced in response to inducing agents such as metals, glucocorticoids, tetracycline, hormones, and the like, are also contemplated for use with the invention. Examples of inducible promoters include, but are not limited to, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; an estrogen receptor; an estrogen receptor fusion; an estrogen analog; IPTG; and the like. Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a reversible promoter. Suitable reversible promoters, including reversible inducible promoters are known in the art. Such reversible promoters may be isolated and derived from many organisms. Such reversible promoters may be isolated and derived from many organisms, e.g., eukaryotes and prokaryotes. Modification of reversible promoters derived from a first organism for use in a second organism is well known in the art. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR)), tetracycline regulated promoters, (e.g., promoter systems including TetActivators, TetON, TetOFF), steroid regulated promoters (e.g., rat glucocorticoid receptor promoter systems, human estrogen receptor promoter systems, retinoid promoter systems, thyroid promoter systems, ecdysone promoter systems, mifepristone promoter systems), metal regulated promoters (e.g., metallothionein promoter systems), pathogenesis-related regulated promoters (e.g., salicylic acid regulated promoters, ethylene regulated promoters, benzothiadiazole regulated promoters), temperature regulated promoters (e.g., heat shock inducible promoters (e.g., HSP-70, HSP-90, soybean heat shock promoter), light regulated promoters, synthetic inducible promoters, and the like.

The vectors of the present disclosure may direct the expression of the nucleic acid in a particular cell type (e.g., tissue-specific regulatory elements are used to express the nucleic acid). Such regulatory elements include promoters that may be tissue/organ specific or cell specific. The term “tissue specific” as it applies to a promoter refers to a promoter that is capable of directing selective expression of a nucleotide sequence of interest to a specific type of tissue in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Tissue specific promoters are known in the art, see for example, Grunennvaldt, et al., Research Journal of Biological Sciences 10 (1-2): 1-9, 2015 and Ali and Kim, Front Plant Sci. 2019 Nov. 1;10:1433, each incorporated herein by reference in its entirety, for exemplary tissue and cell specific promoters identified and characterized for use in plants. Cell type specificity of a promoter may be assessed using methods well known in the art, e.g., immunohistochemical staining.

Thus, it will be appreciated that the present disclosure includes the use of any promoter/regulatory sequence known in the art that is capable of driving expression of the system component(s) operably linked thereto.

The present system or components thereof may be delivered to a cell by any suitable means. In certain embodiments, the system is delivered in vivo. In other embodiments, the system is delivered to isolated/cultured cells in vitro or ex vivo

### 3. METHODS

The present disclosure further provides methods for degrading a protein of interest in a cell. The methods comprise contacting the cell with a system as disclosed herein, wherein the system induces degradation of the protein of interest. In some embodiments, the cell is a non-mammalian cell (e.g., a prokaryotic cell, a plant cell, an insect cell, a fungal cell).

In some embodiments, the cell is in vitro. In some embodiment, the cell is ex vivo. In some embodiments, the cell is in vivo and the methods comprises administering the system to a host organism.

Nucleic acids according to the present disclosure can be transformed, transfected, or otherwise introduced into a wide variety of cells. Transfection refers to the taking up of a nucleic acid by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the system can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle-and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1;459(1-2):70-83), incorporated herein by reference.

In select embodiments, the cell is a plant cell. As such, the systems described herein may be used to engineer a plant or plant cell. The methods comprise providing the disclosed systems to a plant, or a plant cell, seed, fruit, plant part, or propagation material of the plant. The present disclosure provides for a modified plant cells produced by the present system, a plant comprising the plant cell, and a seed, fruit, plant part, or propagation material of the plant. Modified or engineered plant cells of the present disclosure may be as populations of cells, or as a tissue, seed, whole plant, stem, fruit, leaf, root, flower, stem, tuber, grain, animal feed, a field of plants, and the like. Also provided by the present disclosure are modified plant cells, tissues, plants, and products that contain the modified plant cells. In one embodiment, the modified cells, and tissues and products comprise a nucleic acid integrated into the genome, and production by plant cells of a gene product due to the modification.

The present systems and methods have broad applications in gene discovery and validation, mutational and cisgenic breeding, and hybrid breeding. The present systems and methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g. bacterial, fungal, and viral) resistance, high yield, and superior quality. The present systems and methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29;70:667-69), incorporated herein by reference.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (e.g., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. Sec., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In some embodiments, the present systems may be adapted to use in plants. In one embodiment, a series of plant-specific vectors are provided for expression of the present system in plants. The vectors may be optimized for transient expression of the present system in plant protoplasts, or for stable integration and expression in intact plants via the Agrobacterium-mediated transformation.

In some embodiments, the present system may be stably integrated into the plant genome, for example via Agrobacterium-mediated transformation. Thereafter, one or more components of the present system (e.g., the transgene) may be removed by genetic cross and segregation, which may lead to the production of non-transgenic, but genetically modified plants or crops.

Target plants and plant cells include, but are not limited to, monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rapeseed); industrial crops (e.g., bamboo, hemp); medicinal crop (e.g., poppy, foxglove, dahlias, cinchona, digitalis, chili, willow) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea.

### 4. KITS OR SYSTEMS

In another aspect, the disclosure provides kits comprising: one or more components of the disclosed systems. Kits optionally may provide additional components such as buffers or buffer constituents, controls, transformation reagents, cells, and interpretive information.

The kits can also comprise other agents and/or products co-packaged, co-formulated, and/or co-delivered with other components. The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein.

The kits provided herein are in suitable packaging. Suitable packaging includes, but is not limited to, vials, bottles, jars, flexible packaging, and the like. Individual member components of the kits may be physically packaged together or separately.

### 5. EXAMPLES

The accompanying Examples are offered as illustrative as a partial scope and particular embodiments of the disclosure and are not meant to be limiting of the scope of the disclosure.

### Example 1

### Plant Degron System Design Based on SspH1

The system harnesses the E3 catalytic activity of Salmonella SspH1 and the cognate SspH1-interacting domain of its human target, the serine/threonine protein kinase PKN1. SspH1 is a Novel E3 Ligase (NEL) that contains a Leucine-Rich Repeat (LRR) domain for PKN1 recruitment and an NEL catalytic domain for ubiquitination. Specifically, the LRR domain from SspH1 binds to the Homology Region 1b (HR 1b) domain of PKN1. The HR1b-binding surface in the LRR domain is comprised of a concave face from 10 parallel β-sheets, and the specificity of this interaction results from 13 (out of 15) unique contact residues in the LRR which are not present in closely-related NEL domain proteins. This and the fact that SspH1 evolved to target a human protein, suggest high levels of specificity, and imply that LRR is unlikely to recognize an endogenous protein in plants. Moreover, this interaction also occurs in vitro, even between the LRR and HR 1b domains alone, which indicates that this interaction is robust and may be efficient in a synthetic-protein context. Remarkably, the HR1b domain can be ubiquitinated in vitro by a fragment of SspH1 containing only the LRR and NEL domains (LRR-NEL).

The disclosed targeted protein degron design comprises a synthetic biology approach that harnesses the E3 catalytic activity of the SspH1^{LRR-NEL} fragment to target ubiquitination of a POI fused to the HR1b domain (FIG. 1A). To test the functionality of each module in plants, tagged protein fusions were generated for transient expression. The LRR-NEL fragment was fused to a hemagglutinin (HA) tag and the mCherry fluorescent protein at the N and C-termini, respectively, to generate a HA-LRR-NEL-mCherry E3 ligase (herein referred to as NEL E3 ligase chimera for clarity). The HR1b domain was fused to GFP for visualization of target degradation (FIG. 1B). Given that a longer fragment containing HR1a and HR1b resulted in higher order ubiquitination in vitro, HR1b was also tested in combination with HR1a in GFP fusions. Mutations in C492, which is part of the NEL domain catalytic site, renders SspH1 inactive, and alanine (A) substitutions in R181 and R185 of PKN1 abolish its interaction with SspH1. The corresponding mutations were generated to be used as negative controls including HA-LRR-NEL^{C492A}-mCherry (herein referred to as NEL^{C492A} E3 ligase) and non-interacting HR1b^{R181/185A}-GFP (FIG. 1B). These constructs were expressed independently in transient assays in Nicotiana bethamiana under the control of an UBIQUITIN10 promoter to test for their stability in plant cells. The NEL E3 ligase chimera, and both C- and N-terminal GFP fusions of HR1b were stable and accumulated in the cytosol and nucleus in a similar pattern as free GFP (FIG. 1C). Degron fusions that included both HR1a and HR1b resulted in punctate patterns in the cytosol indicating possible protein aggregation (FIG. 1C) and these constructs were not used further.

### Example 2

### The NEL E3 Ligase Fusion Targets HR1b-GFP for Degradation in Plants

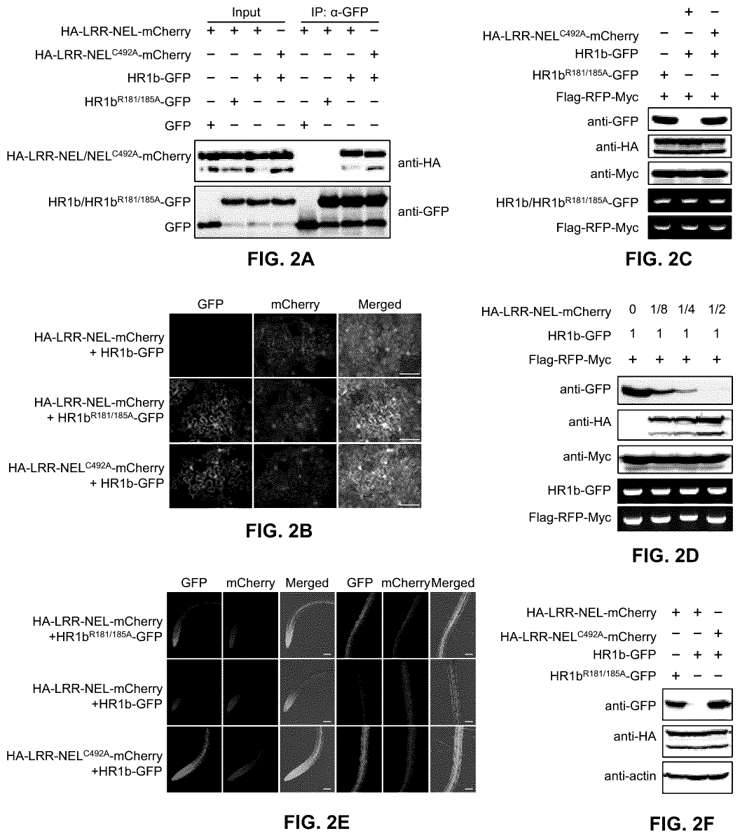

Whether the NEL E3 ligase chimera interacted with HR1b-GFP in plants was first tested using co-immunoprecipitation (co-IP) analysis with crude extracts from infiltrated leaves. Since the stability of the HR1b fusions were likely dependent on the presence of the NEL E3 ligase chimera, leaves were transformed with each construct separately and protein extracts were mixed to test for protein-protein interactions. As shown in FIG. 2A, HR1b-GFP interacts with the catalytically active and inactive forms of the NEL E3 ligase chimera (HA-LRR-NEL-mCherry fusion and HA-LRR-NEL^{C492A}-mCherry). In contrast, the HR1b^{R181/185A} mutations abolished the LRR-HR1b binding, which supports the specificity of the interaction between HR 1b-GFP and the NEL E3 ligase chimera in plant cells. Confocal microscopy was used to test whether expression of both HR1b-GFP and the NEL E3 ligase chimera would result in protein degradation. Co-expression of the NEL E3 ligase chimera and HR1b-GFP resulted in very low accumulation of HR1b-GFP in tobacco epidermal cells (FIG. 2B). In contrast, HR1b-GFP was stable when co-expressed with the catalytically inactive NEL^{C492A} E3 ligase, indicating that the accumulation of HR1b-GFP is sensitive to NEL enzyme activity. The degradation of HR1b-GFP is dependent on its interaction with the LRR domain in planta because the HR1b^{R181/185A}-GFP fusion is stable when co-expressed with the active NEL E3 ligase chimera (FIG. 2B). Immunoblot was used to confirm that the levels of fluorescence from HR1b-GFP reflected protein abundance instead of improper GFP folding. Similar to the confocal analysis, when the NEL E3 ligase chimera is co-expressed with either HR1b-GFP or HR1b^{R181/185A}-GFP, only the mutant HR1b^{R181/185A}-GFP was detected by immunoblotting. HR1b-GFP was again detected when co-expressed with the catalytically inactive NEL^{C492A} E3 ligase. RT-PCR experiments showed that HR1b-GFP and HR1b^{R181/185A}-GFP constructs were transcribed efficiently (FIG. 2C), which supported the conclusion that the NEL E3 ligase chimera targets HR1b-GFP for degradation in co-expression experiments. Co-expression with a Flag-RFP-Myc construct was used as an internal expression control (FIG. 2C). To gain further evidence that the NEL E3 ligase chimera targets HR1b-GFP in plants, a dose-dependent expression analysis was performed by co-infiltration of equal amounts of Agrobacterium tumefaciens carrying the HR1b-GFP construct with increasing amounts of A. tumefaciens carrying the HA-LRR-NEL-mCherry construct. Samples were collected 3 days after infiltration and subjected to western blot. As shown in FIG. 2D, increased expression of the NEL E3 ligase chimera resulted in reduced accumulation of HR1b-GFP. Collectively, these results indicate that HR1b-GFP can be efficiently targeted for degradation by the NEL E3 ligase chimera in Nicotiana transient assays.

To test whether the degron system works efficiently in Arabidopsis, stable-expression constructs of HR1b-GFP, HR1b^{R181/185A}-GFP, the NEL E3 ligase chimera and the catalytically inactive NEL^{C492A} E3 ligase, were generated under the control of the Arabidopsis UBIQUITIN 10 (UBQ10) promoter. Constructs were transformed alone or in combination into wild-type Col-0 plants, and plant roots were imaged by confocal microscopy (FIG. 2E). Similar to the transient assays, very weak fluorescence was detected from HR 1b-GFP in roots that expressed the NEL E3 ligase chimera. Strong fluorescence was detected from HR1b-GFP in roots that expressed the catalytically inactive NEL^{C492A} E3 ligase. Finally, strong GFP fluorescence was observed from lines carrying the HR1b^{R181/185A}-GFP mutant degron together with the catalytically active NEL E3 ligase chimera (FIG. 2E). Immunoblot experiments with these transgenic lines further demonstrated the efficient depletion of HR1b-GFP when the catalytically active enzyme is present but not with the NEL^{C492A} E3 ligase mutant (FIG. 2F). Taken together, these results suggested that the Salmonella SspH1^{LRR-NEL} fragment can degrade HR1b-GFP in Arabidopsis and the degradation is facilitated by the catalytic activity of the NEL E3 ligase chimera and the HR1b interaction.

### Example 3

### NEL E3 Ligase Chimera Targets Endogenous Proteins in the Nucleus and Cytosol

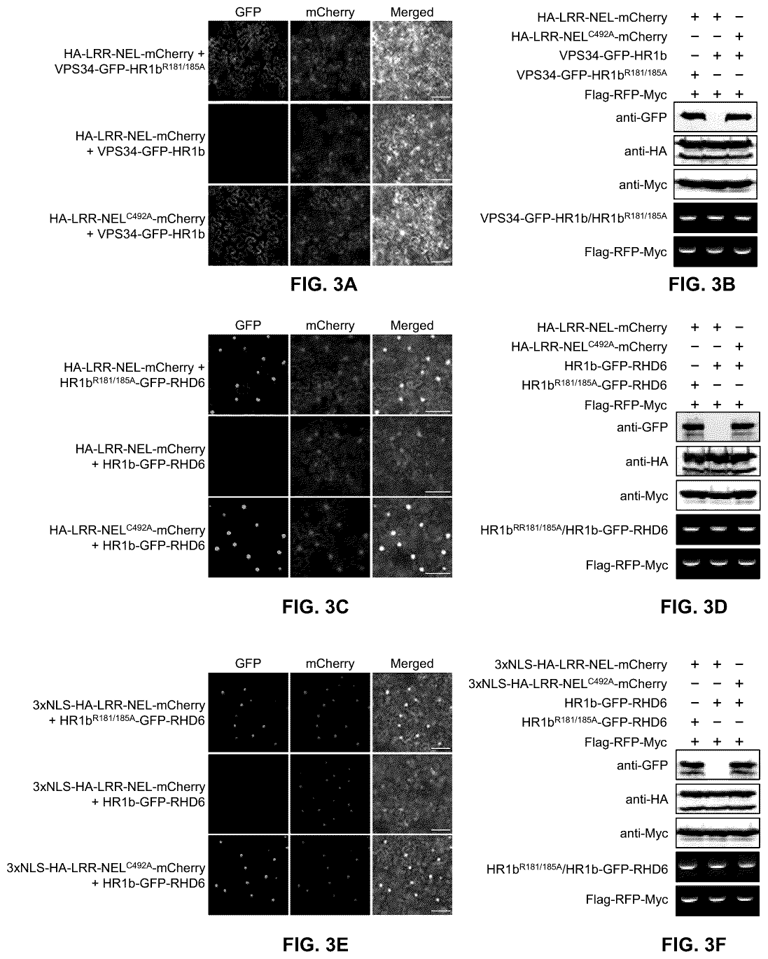

To test whether the NEL E3 ligase chimera can target HR1b fusions to endogenous proteins that localize to the nucleus or the cytosol, HR1b or the mutant degron HR1b^{R181/185A} was fused to the Arabidopsis cytoplasmic protein VPS34 and the transcription factor RHD6. VPS34 encodes Phosphatidylinositol 3-Kinase, a protein of 814 amino acids that functions in the cytosol, while RHD6 is a small transcription factor that is localized to the nucleus. These fusions were transiently co-expressed with the NEL E3 ligase chimeras in Nicotiana. Confocal imaging showed that both the VPS34-GFP-HR1b and HR1b-GFP-RHD6 fusions were efficiently degraded by the NEL E3 ligase chimera. Using the catalytically-inactive enzyme (NEL^{C492A} E3 ligase) or the mutant HR1b^{R181/185A} degron resulted in stable target proteins as expected (FIGS. 3A-C), and these results were validated by western blot (FIGS. 3B, D). The effective degradation of VPS34-GFP-HR1b indicated that HR1b can target large proteins for degradation. A triple nuclear-localization sequence (NLS) was fused to the N-terminus of the NEL E3 ligase chimera and it was co-expressed with HR1b-GFP-RHD6 in Nicotiana to test whether the nucleus-localized NEL E3 ligase chimera could efficiently degrade the transcriptional factor. The HR1b-GFP-RHD6 was again degraded by the nuclear-localized NEL E3 ligase chimera (3×NLS-HA-LRR-NEL-mCherry) in a similar fashion (FIGS. 3E-F). These results indicate that NEL E3 ligase chimera/HR1b system can be used to efficiently target endogenous proteins of different sizes in plants, either in the nucleus or cytosol.

### Example 4

### Establishment of an Inducible Plant Protein Degron System

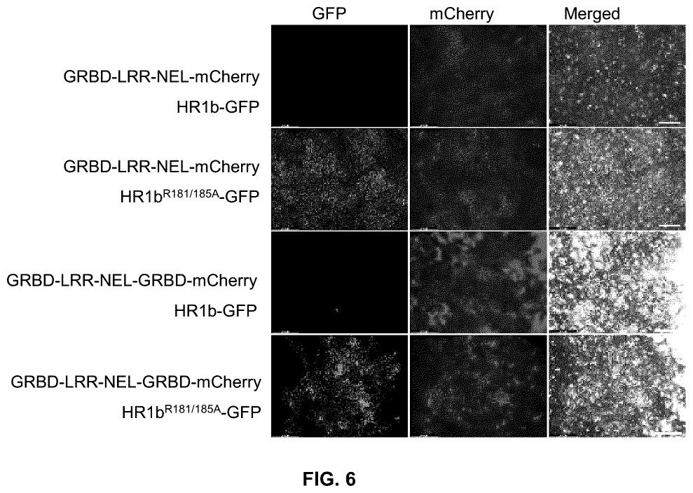

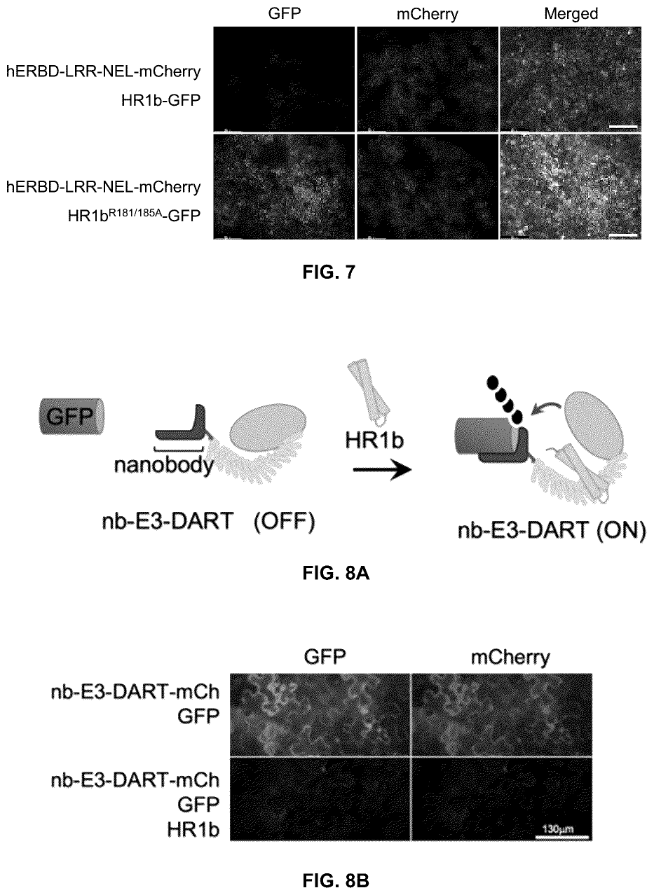

A first attempt to establish the inducibility of target protein degradation was made by harnessing the mode of action of steroid hormone receptors which have been well-exploited in plants. In the absence of hormone, ligand binding domains (LBDs) of steroid hormone receptors are bound by a cytosolic inhibitory complex composed of heat shock proteins 90 (HSP90), HSP70, P23 and other components. This large complex dissociates upon hormone binding to the LBDs, which makes these domains chemically inducible, and therefore, LBDs have been used as regulators in cis of other protein domains including nuclear targeting signals and enzymatic catalytic sites. Initially tested was whether steroid hormone binding domains could be used to control the interaction between an NEL E3 ligase chimera and HR1b-GFP. The ligand binding domain of the glucocorticoid receptor (GRBD) was fused to the LRR-NEL-mCherry fusion and tested whether DEX treatment could be used to control degradation of HR1b-GFP. As shown in FIG. 6, the HR1b-GFP was degraded by the GRBD-LRR-NEL-mCherry fusion in the absence of DEX, suggesting that HR1b binding to the LRR domain is not precluded by the HSP inhibitory complex that binds to GRBD. An additional GRBD domain at the C terminus of the E3 ligase was also insufficient (FIG. 6), and similar fusions between the human estrogen receptor ligand binding domain hERBD and the NEL E3 ligase were also insufficient to block the targeting of HR1b-GFP (FIG. 7). It was concluded that ligand binding domains of steroid hormone receptors are not suitable for controlling the inducibility of the plant degron at the post-translational level.

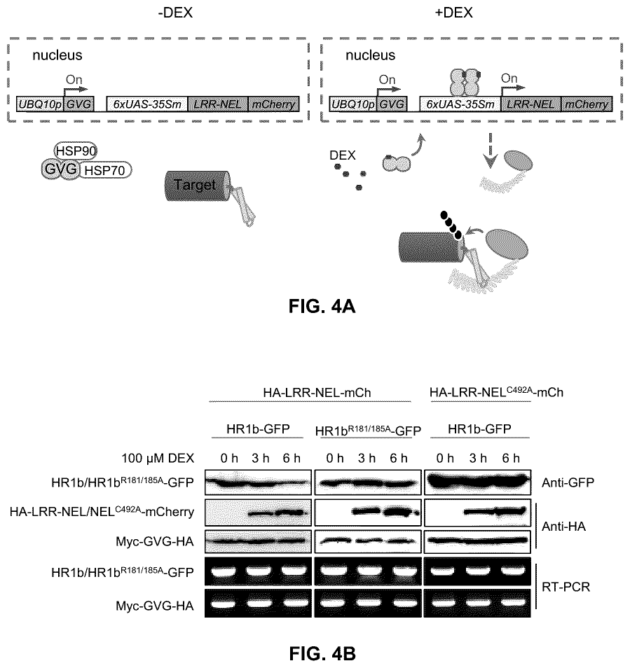

LBDs from the human estrogen receptor (ER) which binds estradiol and the rat glucocorticoid receptor (GR) which binds dexamethasone, have been used successfully in plants for controlled gene expression. To realize the inducible degradation of HR1b-fusions, a transcriptionally-induced degron system was developed by taking advantage of the GAL4-VP16-GR (GVG) transcription induction system to control the expression of the NEL E3 ligase chimera with Dexamethasome (FIG. 4A). In this system, expression of the NEL E3 ligase chimera is driven by a promoter containing six copies of the GAL4 upstream activating sequences (6×UAS) which are bound by the GAL4 DNA binding domain of the GVG chimeric transcription factor. Dex treatment triggers GVG nuclear targeting and transcription of the downstream gene encoding the NEL E3 ligase (FIG. 4A). The GVG-based HA-LRR-NEL-mCherry inducible expression cassette and the HR1b-GFP expression cassette were cloned in tandem into the same vector and then transiently expressed in tobacco. Time-course DEX treatments with the agroinfiltrated tobacco leaves were used to assess the induction of the NEL E3 ligase chimera and the stability of HR1b targets by immunoblotting (FIG. 4B). RT-PCR from infiltrated tissues was used to confirm similar levels of expression of the GVG transcription factor and the HR1b fusions in all samples. When the inducible NEL E3 ligase chimera was co-expressed with HR1b-GFP (lanes 1-3), the E3 ligase fusion was detected 3 and 6 h after DEX treatment and this increase was concomitant with a decrease in HR1b-GFP levels. This decrease suggested induced HR1b-GFP degradation as the result of Dex treatment. This conclusion was supported by the insensitivity of the mutant HR1b^{R181/185A}-GFP fusion at the same time points (lanes 4-6). When the inducible version of the inactive NEL^{C492A} E3 ligase chimera was co-expressed with HR1b-GFP, there was no significant change in GFP abundance even after 6 h of Dex treatment (lanes 7-9). These results demonstrate that DEX-induced plant degron allows the chemically-controlled depletion of HR1b-fusions by the NEL E3 ligase chimera and this is dependent on the LRR-HR1b interaction.

### Example 5

### Rapid Degradation of Plant Proteins by a Dex-Inducible NEL E3 Ligase

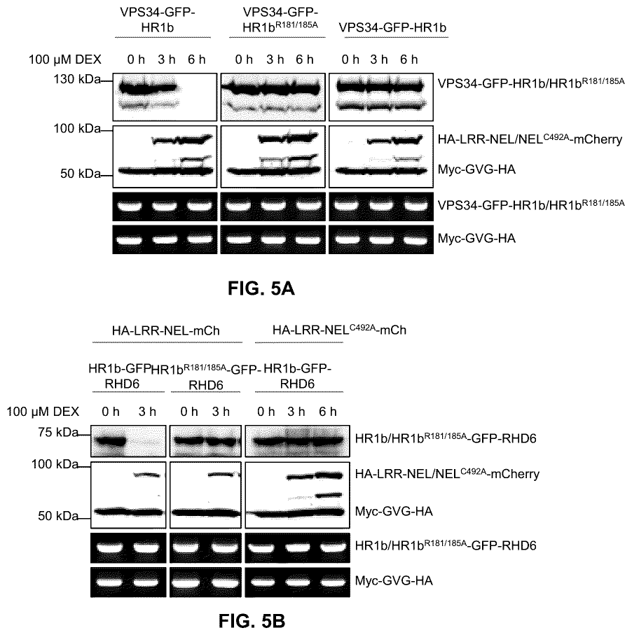

To test the efficiency of the inducible degron when HR1b is fused to native proteins in different locations, the expression cassettes for VPS34-GFP-HR1b and HR1b-GFP-RHD6 fusions were cloned downstream of the inducible HA-LRR-NEL-mCherry expression cassettes in the sample plasmid. DEX treatment of Nicotiana leaves agroinfiltrated with constructs containing NEL E3 ligase fusions and the VPS34-GFP-HR1b construct showed induction of the NEL E3 ligase chimera at 3 and 6 h with concomitant and almost complete depletion of the VPS34 fusion (lanes 1-3). Again, this is most likely due to protein degradation because the VPS34 fused to the mutant HR1b^{R181/185A} fragment was unaffected by Dex treatment even in the presence of the NEL E3 ligase chimera (lanes 4-6). Moreover, the VPS34-GFP-HR1b was stable when the catalytically inactive NEL^{C492A} E3 ligase chimera was induced by Dex (lanes 7-9). Faster inducible degradation was detected with the nuclear fusion protein HR 1b-GFP-RHD6 (FIG. 5B). When this protein was expressed together with the inducible NEL E3 ligase chimera, 3 h of DEX treatment were sufficient to deplete the HR1b-GFP-RHD6 protein (lanes 1-2), demonstrating the rapid and robust degradation of HR1b-tagged proteins by the inducible degron system. By contrast, neither the mutant degron fragment (HR1b^{R181/185A}) nor the catalytically inactive NEL^{C492A} E3 ligase chimera resulted in degradation of RHD6 fusion targets (lanes 3-7). These results combined demonstrate the feasibility of the Dex-inducible degron system to target plant protein degradation with strong specificity and with proteins of varying sizes that function either the nucleus or cytosol.

### Example 6

### E3-DART Activation by the HR1b Degron Peptide

An inducible degron system that targets GFP would have immediate impact on the plant community as many GFP-tagged protein-of-interest fusions that complement loss of function mutants are available. Ubiquitination of such GFP fusions (without HR1b) could in practice be achieved by fusions of the E3-DART ligase to an anti-GFP nanobody (e.g., VhhGFP4) so that target recruitment is mediated exclusively by the GFP-anti-GFP nanobody interaction and the LRR-HR1b interaction is bypassed. However, fusion of the VhhGFP4 nanobody to E3-DART is insufficient for targeting of GFP for degradation in plants (FIG. 8 B, top). Without being bound by theory, this may be due to SspHI and other IpaH family bacterial E3 ubiquitin ligases seem to be autoinhibited by intramolecular interactions. Binding of SspHI to its substrate PKN1 seem to relieve autoinhibition as binding of the HR1b domain from PKN1 overlaps with the SspHI intramolecular binding surface. A system was designed in which the HR1b peptide activated a nanobody-containing E3-DART ligase in trans (FIG. 8A) in such a way that this putative regulatory mechanism could be exploited. Experiments in Nicotiana showed that co-expression of VhhGFP4 nanobody (nb)-E3-DART and HR1b indeed target free GFP for degradation (FIG. 8B, bottom), providing a direct mode of control for the E3 ligase with this small peptide. The nanobody-E3-DART ligase can be activated by delivery of the 78 aa HR1b peptide via transgene expression, incubation with recombinant protein or directly with a synthetic peptide.

### 6. MATERIALS AND METHODS

Plant materials and growth conditions Arabidopsis thaliana plants were grown as previously described (Zheng et al., 2014, plant signaling & behavior 9, e972113), and the ecotype Columbia (Col-0) was used as wild-type control. All the Arabidopsis transformations were performed by floral dip using Agrobacterium tumefaciens stain C58C1. Nicotiana benthamiana plants were grown in soil in the lab under a 16-h light/8-h dark photoperiod for 40 days and the abaxial epidermis of Nicotiana benthamiana leaves were infiltrated with bacteria suspensions as previously reported (Chen et al., 2008, Plant Physiol 146, 368-376).

Chemical treatments Dexamethasone was purchased from SIGMA-Aldrich (Cat. No. D1756). Leaves were excised below the petiole 72 h after agroinfiltration and immediately placed in a solution of 0.5×Murashige and Skoog media (MS) containing 100 μM Dexamethasone (DEX) for the time indicated.

Plasmid construction All plasmids used were generated by the GoldenBraid system or obtained from other sources. Each DNA fragment was domesticated using the GoldenBraid domestication tool (gbcloning.upv.es/tools/domestication/) for either PCR amplification or gene synthesis before cloning into the entry vector pUPD2. The coding sequences of the LRR-NEL fragment (E162-N700) of Salmonella enterica serovar Typhimurium effector protein SspH1, HR1a-1b domains (residues W13-P199) and HR1b domain (residues A122-P199) of human protein kinase N1 (PKN1) and the anti-GFP nanobodies vhhGFP4 Kless (lysine-less) and GS2 were codon-optimized for expression in plants and synthesized (Integrated DNA Technologies) before cloning. The Flag-tagged ligand binding domain of rat glucocorticoid receptor (GRBD, residues E508-K795) was amplified from the pSW610-GR-LhG4_BD plasmid by introducing the coding sequence of triple Flag tags into the forward primer, while the Flag-tagged ligand binding domain of human estrogen receptor (hERBD, residues S292-V595), was synthesized.

Starting fragments were cloned into pUPD2 using BsmBI and T4 DNA ligase to generate entry clones for most constructs. Site directed mutagenesis or nested PCR were used to generate pUPD2 entry plasmids containing mutant LRR-NEL^{C492A} or HR1b^{R181/185A} fragments. When used, a Gly/Ser linker was introduced as part of either the forward primer or the reverse primer of a GBpart before cloning into pUPD2. To generate the Dex-inducible expression cassettes, the coding sequences of the chimeric transcription factor GAL4-VP16-GR (GVG) were codon-optimized and synthesized with an N-terminal Myc tag and a C-terminal Hemagglutinin tag. The tE9-6xUAS-35Smini fragment was synthesized directly and all parts were cloned into pUPD2 with BsmBI, forming the pUPD2-Myc-GVG-HA-tE9-6xUAS-35Smini plasmid.

All transcriptional units (or transgenes) were cloned first in the pGDB3_alpha1 vector. pGDB3_alpha1 plasmids were used for transient expression of a single transgene. GoldenBraid-based ligation reactions from alpha-level plasmids or above were used to combine two or three transcriptional units into pDGB3_omega1 or pDGB3_alpha1, respectively. The basta resistance expression cassette was amplified from pTNos: BASTA: tNos plasmid (GB0023) and cloned into pDGB3_alpha2 with BsmBI, creating TNOSₚᵣₒ:BASTA plasmid. Each transcriptional unit in the alpha1-level degron plasmids above was then inserted into pDGB3_omega1 with basta resistance cassette from Nosₚᵣₒ:BASTA plasmid, while the transcription unit of each target plasmid was assembled into pDGB3_omega1 with kanamycin resistance cassette from pEGB Pnos:NptII:Tnos (GB0184) plasmid. Escherichia coli stain GC5 was used for the construction and propagation of all plasmids, and all the generated alpha1-level and omega1-level plasmids were transformed into Agrobacterium tumefaciens stain C58C1.

Co-immunoprecipitation Assay Immunoprecipitation assays were conducted with crude extracts from agroinfiltrated Nicotiana leaves with overexpressed HA-LRR-NEL-mCherry, HA-LRR-NEL^{C492A}-mcherry, HR1b-GFP, and HR1b^{R181/185A}-GFP and GFP. Total proteins were extracted 72 hours post-agroinfiltration with native buffer (50 mM Tris-MES pH8.0, 0.5 M sucrose, 1 mM MgCl₂, 10 mM EDTA, 5 mM DTT, and protease inhibitor cocktail cOmplete Mini tablets (Roche). The supernatants were collected for Co-IP assay after centrifugation at 18 000 g at 4° C. three times for 5 min each time. In brief, equal amounts of HA-LRR-NEL-mCherry and HA-LRR-NEL^{C492A}-mcherry were mixed with either HR1b-GFP or HR1b^{R181AR185A}-GFP and incubated with 25 μL of the agarose-conjugated anti-GFP monoclonal antibody (D153-8; MBL) at 4° C. for 4 h. After washing three times with PBS buffer (pH 7.4), the bead-bound proteins were eluted in sodium dodecyl sulfate (SDS) sample buffer and then subjected to SDS-polyacrylamide gel electrophoresis (PAGE) for immunoblotting analysis. The HA-tagged proteins were detected with monoclonal anti-HA antibody (H9658; Sigma-Aldrich) at a dilution of 1:10000 while the GFP-tagged proteins were detected with polyclonal anti-GFP antibody (SAB4301138; Sigma-Aldrich) at a dilution of 1:3000.

Protein degradation analysis For the in vivo protein degradation assay in tobacco, the omegal-level destination plasmids harboring different combinations of HA-tagged E3 ligase chimeras and GFP-tagged targets expression cassettes and Flag-RFP-Myc overexpression plasmid were co-infiltrated into Nicotiana leaves. Samples were harvested 3 days after infiltration and total proteins were extracted followed by the immunoblotting with monoclonal anti-GFP antibody (11814460001; Sigma-Aldrich) at 1:3000 dilution and anti-HA antibody (H9658; Sigma-Aldrich) at 1:10000 dilution. The Flag-RFP-Myc was used as the internal expression control and detected with monoclonal anti-Myc (M4439; Sigma-Aldrich) at 1:5000 dilution. The transcriptional levels of the GFP-tagged HR1b and HR1b^{R181/185A} fusion constructs were analyzed by semi-quantitative RT-PCR. For inducible degradation of target proteins, Nicotiana leaves infiltrated with different omegal-level destination plasmids harboring the Myc-GVG-HA expression cassette, the HA-tagged E3 ligase chimeras inducible expression cassettes, and GFP-tagged targets expression cassettes were treated with 100 μM DEX and collected at different time points for degradation analysis. The Myc-GVG-HA was used as internal control.

For analysis of protein stability in Arabidopsis, 10-day-old seedlings of mCherry- and GFP-tagged double marker lines grown on ½ MS medium were collected for protein extraction with native extraction buffer. The protein levels were detected with either anti-GFP or anti-HA monoclonal antibodies, and endogenous Actin were determined using rabbit anti-Actin polyclonal antibody (ab 197345; Abcam) at 1:5000 dilution.

Microscopy Epifluorescence imaging of infiltrated Nicotiana leaves was carried out in a Leica DM5000 compound microscope equipped with a GFP filter cube (EX 470/40 EM 525/50) and TX2 filter cube (Ex 560/40 Em BP645/75) using a HCX PL APO 20×/0.70 objective. Images were captured using a Leica DFC365 FX monochrome digital camera. Confocal imaging was done in a Zeiss LSM 710 or LSM980 at the Cellular and Molecular Imaging Facility at North Carolina State University. Confocal imaging for GFP and mCherry were performed at the excitation wavelengths of 488 nm and 561 nm, respectively.

7. Sequences

SEQ ID NO: 1
EYDAVWSKWERDAPAGESPGRAAVVQEMRDCLNNGNPVLNVGASGLTTLPDRLPPHI
TTLVIPDNNLTSLPELPEGLRELEVSGNLQLTSLPSLPQGLQKLWAYNNWLASLPTLPPG
LGDLAVSNNQLTSLPEMPPALRELRVSGNNLTSLPALPSGLQKLWAYNNRLTSLPEMSP
GLQELDVSHNQLTRLPQSLTGLSSAARVYLDGNPLSVRTLQALRDIIGHSGIRIHFDMAG
PSVPREARALHLAVADWLTSAREGEAAQADRWQAFGLEDNAAAFSLVLDRLRETENFK
KDAGFKAQISSWLTQLAEDAALRAKTFAMATEATSTCEDRVTHALHQMNNVQLVHNA
EKGEYDNNLQGLVSTGREMFRLATLEQIAREKAGTLALVDDVEVYLAFQNKLKESLEL
TSVTSEMRFFDVSGVTVSDLQAAELQVKTAENSGFSKWILQWGPLHSVLERKVPERFNA
LREKQISDYEDTYRKLYDEVLKSSGLVDDTDAERTIGVSAMDSAKKEFLDGLRALVDEV
LGSYLTARWRLN
SEQ ID NO: 2
ATNLSRVAGLEKQLAIELKVKQGAENMIQTYSNGSTKDRKLLLTAQQMLQDSKTKIDII
RMQLRRALQAGQLENQAAP

What is claimed is:

1. A system for targeted protein degradation in a cell comprising:
a first polypeptide comprising Leucine-Rich Repeat (LRR) domain and novel E3 ligase (NEL) catalytic domain of SspH1, or a nucleic acid encoding thereof; and
a second polypeptide comprising Homology Region 1b (HR1b) domain of human PKN1, or a nucleic acid encoding thereof.

2. The system of claim 1, wherein the first polypeptide or the second polypeptide further comprise a targeting moiety configured to specifically bind a protein of interest.

3. The system of claim 2, wherein the targeting moiety directly or indirectly binds the protein of interest.

4. The system of claim 3, wherein the targeting moiety is an antibody or fragment thereof to the protein of interest.

5. The system of claim 3, wherein the targeting moiety is a first part of a specific binding pair and the protein of interest comprises a second part of the specific binding pair.

6. The system of claim 1, wherein the first polypeptide or the second polypeptide is linked to a third polypeptide comprising the protein of interest.

7. The system of claim 1, wherein the nucleic acid encoding the first polypeptide and/or the nucleic acid encoding the second polypeptide are operably linked to a tissue or cell specific promoter and/or a regulatable promoter.

8. The system of claim 1, wherein the first polypeptide or second polypeptide are heterologous to the cell.

9. The system of claim 1, wherein the cell is a non-mammalian cell.

10. The system of claim 1, wherein the cell is a plant cell.

11. The system of claim 1, wherein the protein of interest is a membrane protein, a receptor, a hormone, a transport protein, a transcription factor, a cytoskeletal protein, an extracellular matrix protein, a signal-transduction protein, or an enzyme.

12. A cell comprising a system of claim 1.

13. A plant comprising a system of claim 1 or a cell comprising the system.

14. A fusion protein comprising:
a protein of interest; and
Homology Region 1b (HR1b) domain of human PKN1 or a polypeptide comprising Leucine-Rich Repeat (LRR) domain and novel E3 ligase (NEL) catalytic domain of SspH1.

15. The fusion protein of claim 14, wherein the protein of interest is linked to the Homology Region 1b (HR1b) domain of human PKN1 or a polypeptide comprising Leucine-Rich Repeat (LRR) domain and novel E3 ligase (NEL) catalytic domain of SspH1 by an amino acid linker.

16. A method for degrading a protein of interest in a cell, comprising contacting the cell with a system of claim 1, wherein the system induces degradation of the protein of interest.

17. The method of claim 16, wherein the cell is a non-mammalian cell.

18. The method of claim 16, wherein the cell is a plant cell.

19. The method of claim 16, wherein the cell is in vitro, ex vivo, or in vivo.

20. The method of claim 19, wherein the method comprises administering the system to a host organism.
